SEQUENCE LISTING (7 sequences)

SEQ ID NO: 1 - protein, 10 aa, Homo sapiens
GYTFTSCVMN

SEQ ID NO: 2 - protein, 17 aa, Homo sapiens
YINPYNDGTKYNEKFKG

SEQ ID NO: 3 - protein, 10 aa, Homo sapiens
EGFYYGNFDN

SEQ ID NO: 4 - protein, 11 aa, Homo sapiens
RASENIYSYLT

SEQ ID NO: 5 - protein, 7 aa, Homo sapiens
NAKTLPE

SEQ ID NO: 6 - protein, 9 aa, Homo sapiens
QHHYGTPYT

SEQ ID NO: 7 - protein, 10 aa, Homo sapiens
GYTFTSXVMN

(19) United States
(12) Patent Application Publication
(10) Pub. No.: US 2025/0243294 A1
(43) Pub. Date: Jul. 31, 2025

(54) METHODS TO REGULATE GLYCOLYSIS VIA TARGETING EXTRACELLULAR ALPHA-ENOLASE FOR TREATING HUMAN DISEASES

(71) Applicant: HUNILIFE BIOTECHNOLOGY, INC, Taipei City (TW)

(72) Inventors: TA-TUNG YUAN, Taipei City (TW); WEI-CHING HUANG, Taipei City (TW); I-CHE CHUNG, Taipei City (TW)

(21) Appl. No.: 18/701,967

(22) PCT Filed: Nov. 24, 2022
(86) PCT No.: PCT/CN2022/134197

§ 371 Date: Apr. 17, 2024

Related U.S. Application Data
(60) Provisional application No. 63/283,352, filed on Nov. 26, 2021.

(51) Int. Cl. C07K 16/40; A61K 39/00; A61P 11/00; A61P 35/00

(52) U.S. Cl. CPC C07K 16/40; A61P 11/00; A61P 35/00; A61K 2039/505; A61K 2039/545

(57) ABSTRACT

Provided are identifies new methods for regulating (or reprogramming) glycolytic reaction by using alpha-enolase (ENO-1) antagonist or agonist. More specifically, provided are methods for glycolysis reprogramming by targeting extracellular ENO-1 or membrane-associated ENO-1.

### BACKGROUND OF INVENTION

### Field of the Invention

The present disclosure identifies new methods for regulating (or reprogramming) glycolytic reaction by using an alpha-enolase (ENO-1) antagonist or agonist. More specifically, the disclosure relates to methods for glycolysis reprogramming by targeting extracellular ENO-1 or membrane-associated ENO-1.

### Background Art

Glycolysis is one of the main metabolic pathways that provides energy for cellular processes, which comprises multiple enzymes that transform glucose into pyruvate. Among various energy sources, glycolysis was found to be preferentially utilized by different types of cells associated with diseases. For example, highly proliferating cells like cancer cells, compared to normal cells, prefer utilizing glycolysis than other energy sources. In addition, immune cells also preferentially use glycolysis as a source of energy upon immune response. Despite glycolysis is much less efficient than oxidative phosphorylation (OXPHOS), the cellular levels of intermediate metabolites resulting from glycolytic reprogramming facilitate cancer progression and also determine the activation/proliferation/differentiation of immune cells.

Lactate is active metabolite of glycolysis, which often accumulates in tumor microenvironment or the sites of inflammation, reflecting the activity of glycolysis.

The skewed energy programing toward glycolysis is hypothesized to be an important contributor for the pathogenesis of various diseases. Therefore, it is conceivable to design drug modality, antagonist or agonist, based on the principle to regulate energy reprograming of glycolysis, i.e., increasing or decreasing.

As result, if a method for controlling glycolysis may be established, it is possible to control the pathway of cancer progression.

### SUMMARY OF INVENTION

For the abovementioned purpose, the present disclosure discloses methods of regulating glycolysis by targeting extracellular or membrane-associated alpha-enolase (enolase-1, ENO-1) to control (or affect) glycolytic reprogramming, wherein the methods comprise administration of small molecule, peptide, aptamer, antibody, or antibody-based modality which have a binding capacity to ENO-1, e.g., human ENO-1 antibody, as an antigen binding structural domain to induce the antagonistic or agonistic effect of ENO-1. The ENO-1 antagonist or agonist can bind to free ENO-1 and/or cell membrane-associated ENO-1, which has an important application prospect in the treatment of human diseases involving glycolytic reprogramming.

In accordance with certain embodiments of the disclosure, the human diseases or disorders may be any condition arising from aberrant activation or expression of ENO-1 protein. Examples of such diseases include cancers, immune diseases, and fibrotic diseases or a combination thereof.

In accordance with certain embodiments of the disclosure, the cancers which can be treated by using the methods disclosed herein based on antagonistic or agonistic effect on glycolysis mediated by extracellular or cell surface ENO-1 include, but are not limited to, gastrointestinal cancer, such as colon cancer, colorectal cancer, esophagus cancer, gastric cancer, hepatocellular cancer, liver cancer and pancreatic cancer, lymphoproliferative disorders, such as lymphoma, lung cancer, such as non-small cell lung cancer, such as, adenocarcinoma of the lung, squamous carcinoma of the lung, and small-cell lung cancer, blood cancer, such as leukemia, bladder cancer, blastoma, brain cancer, breast cancer, cancer of the peritoneum, cervical cancer, endometrial or uterine carcinoma, glioblastoma, glioma, head and neck cancer, kidney cancer and other neoplasms known in the art.

In accordance with certain embodiments of the disclosure, the immune diseases may include, but are not limited to, multiple sclerosis, systemic sclerosis, systemic lupus erythematosus, rheumatoid arthritis, type 1 diabetes, atherosclerosis, inflammatory bowel disease, macrophage activation syndrome, atopic dermatitis, and psoriasis.

In accordance with certain embodiments of the disclosure, the fibrotic diseases or disorder may include idiopathic pulmonary fibrosis (IPF), pulmonary hypertension, pulmonary fibrosis, emphysema, nonalcoholic steatohepatitis, pancreatic fibrosis, intestinal fibrosis, cardiac fibrosis, myelofibrosis, arthrofibrosis, interstitial lung diseases, non-specific interstitial pneumonia (NSIP), usual interstitial pneumonia (UIP), endomyocardial fibrosis, mediastinal fibrosis, retroperitoneal fibrosis, progressive massive fibrosis (a complication of coal workers' pneumoconiosis), nephrogenic systemic fibrosis, Crohn's disease, old myocardial infarction, scleroderma/systemic sclerosis, neurofibromatosis, Hermansky-Pudlak syndrome, diabetic nephropathy, renal fibrosis, hypertrophic cardiomyopathy (HCM), hypertension-related nephropathy, focal segmental glomerulosclerosis (FSGS), radiation-induced fibrosis, uterine leiomyomas (fibroids), alcoholic liver disease, hepatic steatosis, hepatic fibrosis, hepatic cirrhosis, hepatitis C virus (HCV) infection, chronic organ transplant rejection, fibrotic conditions of the skin, keloid scarring, Dupuytren contracture, Ehlers-Danlos syndrome, epidermolysis bullosa dystrophica, oral submucous fibrosis, and fibro-proliferative disorders. In a preferred embodiment, the fibrotic diseases or disorders may include idiopathic pulmonary fibrosis, pulmonary hypertension, emphysema, nonalcoholic steatohepatitis, pancreatic fibrosis, renal fibrosis, intestinal fibrosis, cardiac fibrosis, myelofibrosis, arthrofibrosis, or systemic sclerosis.

In other embodiments, the drug modality, antagonistic or agonistic, targeting ENO-1 can be small molecule, peptide, and aptamers.

In accordance with certain embodiments of the disclosure, the administering is by oral, parenteral, buccal, vaginal, rectal, inhalation, insufflation, sublingual, intramuscular, subcutaneous, topical, intranasal, intraperitoneal, intrathoracic, intravenous, epidural, intrathecal, or intracerebroventricular route, or by injection into joint.

In one embodiment, the administering step is by intravenous bolus injection or intravenous infusion. In another embodiment, the administering step is by subcutaneous bolus injection.

In one embodiment, the administering step is provided with a dosing regimen comprising one or more dosing cycles of the ENO-1 antibody at a fixed dose, e.g., about 10-3000 mg every 2 to 4 weeks.

In accordance with certain embodiments of the disclosure, the subject is a mammal. In a preferred embodiment the subject is human.

Other aspect of the disclosed invention will become apparent with the following description.

### BRIEF DESCRIPTION OF THE DRAWINGS

Embodiments will now be described by way of examples, with reference to the accompanying drawings which are meant to be exemplary and not limiting.

FIG. 1 shows that ENO-1 mAb reduced (A) lactate production (i.e. glycolysis) and (B) cell migration in LPS-stimulated human PBMC.

FIG. 2 shows that ENO-1 mAb reduced (A) lactate production (i.e. glycolysis) and (B) immune cells infiltration in peritoneal lavage fluid from mice of LPS-induced peritonitis model.

FIG. 3 shows that ENO-1 mAb reduced lactate production in human multiple myeloma cell lines (A) RPMI-8226 and (B) U266, indicating inhibition of glycolysis.

FIG. 4 shows that ENO-1 mAb reduced (A) serum levels of lactate and (B) tumor growth in multiple myeloma subcutaneous xenograft model.

FIG. 5 shows that ENO-1 mAb reduced (A) lactate production (i.e. glycolysis) and (B) migration of prostate cancer cell line PC-3 in a dose-dependent manner.

FIG. 6 shows that ENO-1 mAb reduced lactate production in (A) primary human endothelial cells and (B) lung fibroblasts, indicating inhibition of glycolysis.

FIG. 7 shows that ENO-1 mAb reduced lactate production in the (A) lungs and (B) bronchoalveolar lavage fluid (BALF) in a murine model of bleomycin-induced pulmonary fibrosis.

### DESCRIPTION

Reference is made in detail to the embodiments of the present disclosure, examples of which are illustrated in the accompanying drawings. While the invention is described in conjunction with the embodiments below, it is understood that they are not intended to limit the invention to these embodiments and examples. On the contrary, the invention is intended to cover alternatives, modifications and equivalents, which can be included within the spirit and scope of the invention as defined by the appended claims. Furthermore, in the following detailed description of the present invention, numerous specific details are set forth in order to more fully illustrate the present invention. However, it is apparent to one of ordinary skill in the prior art having the benefit of this disclosure that the present invention can be practiced without these specific details. In other instances, well-known methods and procedures, components and processes have not been described in detail so as not to unnecessarily obscure aspects of the present invention. It is, of course, appreciated that in the development of any such actual implementation, numerous implementation-specific decisions must be made in order to achieve the developer's specific goals, such as compliance with application and business-related constraints, and that these specific goals vary from one implementation to another and from one developer to another. Moreover, it is appreciated that such a development effort can be complex and time-consuming but is nevertheless a routine undertaking of engineering for those of ordinary skill in the art having the benefit of this disclosure.

### General Definitions

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press, 1989); DNA Cloning, Volumes I and II (D. N. Glover ed., 1985); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Antibodies: A Laboratory Manual, by Harlow and Lane s (Cold Spring Harbor Laboratory Press, 1988); and Handbook Of Experimental Immunology, Volumes I-IV (D. M. Weir and C. C. Blackwell, eds., 1986).

The terms “antibody” and “immunoglobulin” are used interchangeably in the broadest sense and include monoclonal antibodies (e.g., full length or intact monoclonal antibodies), polyclonal antibodies, monovalent, multivalent antibodies, multi-specific antibodies (e.g., bispecific antibodies so long as they exhibit the desired biological activity) and may also include certain antibody fragments (as described in greater detail herein). An antibody can be chimeric, human, humanized and/or affinity matured.

The antibodies can be full-length or can comprise a fragment (or fragments) of the antibody having an antigen-binding portion, including, but not limited to, Fab, F(ab′)2, Fab′, F(ab)′, Fv, single chain Fv (scFv), bivalent scFv (bi-scFv), trivalent scFv (tri-scFv), Fd, dAb fragment (e.g., Ward et al, Nature, 341:544-546 (1989)), an CDR, diabodies, triabodies, tetrabodies, linear antibodies, single-chain antibody molecules, and multispecific antibodies formed from antibody fragments. Single chain antibodies produced by joining antibody fragments using recombinant methods, or a synthetic linker, are also encompassed by the present invention. Bird et al. Science, 1988, 242:423-426. Huston et al, Proc. Natl. Acad. Sci. USA, 1988, 85:5879-5883.

The terms “cancer” and “cancerous” refer to or describe the physiological condition in mammals that is typically characterized by unregulated cell growth/proliferation. Examples of cancer include, but are not limited to, gastrointestinal cancer, such as colon cancer, colorectal cancer, esophagus cancer, gastric cancer, hepatocellular cancer, liver cancer and pancreatic cancer, lymphoproliferative disorders, such as lymphoma, lung cancer, such as non-small cell lung cancer, such as, adenocarcinoma of the lung, squamous carcinoma of the lung, and small-cell lung cancer, blood cancer, such as leukemia, bladder cancer, blastoma, brain cancer, breast cancer, cancer of the peritoneum, cervical cancer, endometrial or uterine carcinoma, glioblastoma, glioma, head and neck cancer, kidney cancer and other neoplasms known in the art.

As used herein, “treatment” refers to clinical intervention in an attempt to alter the natural course of the individual or cell being treated and can be performed either for prophylaxis or during the course of clinical pathology. Desirable effects of treatment include preventing occurrence or recurrence of disease, alleviation of symptoms, diminishment of any direct or indirect pathological consequences of the disease, preventing or decreasing inflammation and/or tissue/organ damage, decreasing the rate of disease progression, amelioration or palliation of the disease state, and remission or improved prognosis. In some embodiments, antibodies of the invention are used to delay development of a disease or disorder.

An “individual” or a “subject” is a vertebrate. In certain embodiments, the vertebrate is a mammal. Mammals include, but are not limited to, farm animals (such as cows), sport animals, pets (such as cats, dogs, and horses), primates, mice, and rats. In certain embodiments, the vertebrate is a human.

An “effective amount” refers to an amount effective, at dosages and for periods of time necessary, to achieve the desired therapeutic or prophylactic result.

A “therapeutically effective amount” of a substance/molecule of the invention may vary according to factors such as the disease state, age, sex, and weight of the individual, and the ability of the substance/molecule, to elicit a desired response in the individual. A therapeutically effective amount is also one in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

ENO-1 is a multiple functional protein, which was first found in cytosol as a key enzyme of the glycolysis pathways. It has been known that modulation of intracellular or cytosolic ENO-1 is able to affect or reprogram glycolysis by reagents or genetic approaches, indicated by the amount of lactate production.

In contrast to normal cells, ENO-1 is also found to express on the cell surfaces of many cancer cells as a plasminogen receptor and on activated hematopoietic cells, such as neutrophils, lymphocytes, and monocytes. It is known that the up-regulation of plasminogen receptor can induce a cascade response of the urokinase plasminogen activation system and results in extracellular matrix degradation.

The current disclosure described methods to regulate (or reprogram) glycolysis by targeting extracellular or cell membrane-associated ENO-1, instead of targeting intracellular ENO-1 by other approaches.

This is a previously unknown method of regulating glycolysis, which can be used for drug design to treat human diseases by targeting extracellular or cell membrane-associated ENO-1.

For the purpose of the present disclosure, a method for regulating glycolysis in a cell is provided. The method comprises contacting the cell with an alpha-enolase (ENO-1) antagonist or agonist which specifically binds to extracellular or membrane-associated ENO-1 of the cell. In one embodiment, the ENO-1 antagonist or agonist is an ENO-1 antibody or the binding fragment thereof, an anti-ENO-1 peptide, an anti-ENO-1 aptamer or a small molecule compound. The agonist of ENO-1 may be Tamoxifen.

In accordance with embodiments of the disclosure, the ENO-1 antagonist or agonist may be an ENO-1 antibody, e.g., HuL001 as described in US2019/0322762, the contents of which are incorporated by reference in its entirety.

In other embodiments, the ENO-1 antagonist or agonist can be any antibody that binds specifically to extracellular or cell surface ENO-1 in a cell. For example, the ENO-1 antagonist or agonist may be a mouse or humanized anti-ENO-1 antibody, or a scFv or Fab fragment thereof. An exemplary anti-ENO-1 antibody, e.g. HuL001 as described in US2019/0322762, the contents of which are incorporated by reference in its entirety, may comprise a heavy-chain variable domain having three complementary regions including HCDR1 (GYTFTSCVMN; SEQ ID NO: 1), HCDR2 (YINPYNDGTKYNEKFKG; SEQ ID NO: 2) and HCDR3 (EGFYYGNFDN; SEQ ID NO: 3), and a light-chain variable domain having three complementary regions including LCDR1 (RASENIYSYLT; SEQ ID NO: 4), LCDR2 (NAKTLPE; SEQ ID NO: 5) and LCDR3 (QHHYGTPYT; SEQ ID NO: 6). An another exemplary anti-ENO-1 antibody may comprise a heavy-chain variable domain having three complementary regions including HCDR1 (GYTFTSXVMN, wherein X is any amino acid but cysteine; SEQ ID NO: 7), HCDR2 (YINPYNDGTKYNEKFKG; SEQ ID NO: 2) and HCDR3 (EGFYYGNFDN; SEQ ID NO: 3), and a light-chain variable domain having three complementary regions including LCDR1 (RASENIYSYLT; SEQ ID NO: 4), LCDR2 (NAKTLPE; SEQ ID NO: 5) and LCDR3 (QHHYGTPYT; SEQ ID NO: 6).

For another purpose of the present disclosure, a use of an alpha-enolase (ENO-1) antagonist or agonist in manufacturing a medicament for regulating glycolysis is provided. For another purpose of the present disclosure, an ENO-1 antagonist or agonist for use in regulating glycolysis in a cell is provided. For still another purpose of the present disclosure, a method of treating human disease arising from aberrant activation or expression of ENO-1 is provided. The method comprises administering an effective amount of ENO-1 antagonist or agonist targeting extracellular or membrane-associated ENO-1.

In other embodiments, the ENO-1 antagonist or agonist can be any antibody that binds specifically to extracellular or cell surface ENO-1 in a cell. The administering step is by subcutaneous injection, intramuscular injection, intravenous injection, intraperitoneal injection or orthotopic injection, preferably subcutaneous or intravenous injection. In another embodiment, the administering step is by intravenous bolus injection or intravenous infusion. In still another embodiment, the administering step is by subcutaneous bolus injection. The administering step is provided with a dosing regimen comprising one or more dosing cycles of the ENO-1 antagonist or agonist at a fixed dose, e.g. about 10-3000 mg every 2 to 4 weeks, preferably, 80-800 mg every 2 to 4 weeks.

### EXAMPLES

To confirm the regulation of the ENO-1 to the glycolysis, following tests and analyses are performed.

### Example 1. In Vitro and In Vivo Effects of Targeting Extracellular ENO-1 to by Monoclonal Antibody to Regulate Glycolysis in Immune Cells

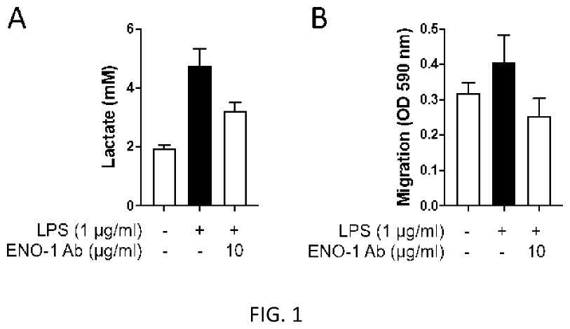

In this example, one method of regulating glycolysis by monoclonal antibody (mAb) targeting extracellular or membrane-associated ENO-1 in immune cells upon inflammation was shown. In FIG. 1, human whole blood was stimulated with 1 μg/ml of lipopolysaccharide (LPS) in the absence or presence of 10 μg/ml of ENO-1 mAb for 4 hours before isolation of peripheral mononuclear cells (PBMCs). PBMCs resuspended in culture medium with 2% of fetal bovine serum (FBS) were subjected to transwells and allowed to migrate for 18 hours to the bottom well which contained culture medium with 10% FBS. Supernatants in the transwells were collected for measurement of lactate production. Results showed targeting extracellular or surface ENO-1 by ENO-1 mAb could reduce LPS-induced lactate production (i.e., glycolysis) as well as cell migration.

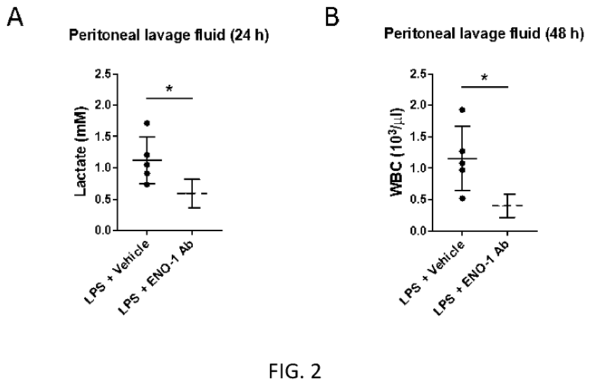

LPS-induced peritonitis in C57BL/6 mice is commonly used as an in vivo experimental inflammatory model. The 8-week-old male C57BL/6 mice were intraperitoneally injected with 10 mg/kg of ENO-1 mAb 2 hours before intra-peritoneal injection of 2 mg/kg of LPS. Peritoneal lavage fluids were collected for analysis of lactate production and immune cells infiltration after 24 and 48 hours of LPS injection, respectively. FIG. 2 illustrates ENO-1 mAb reduces both lactate production (i.e. glycolysis) and immune cells infiltration in the peritoneal lavage fluid from murine model of LPS-induced peritonitis.

### Example 2. In Vitro and In Vivo Effects of Targeting Extracellular ENO-1 by Monoclonal Antibody to Regulate Glycolysis in Multiple Myeloma

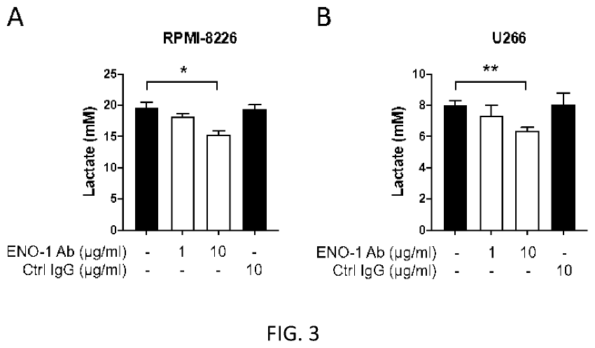

In this example, one method of regulating glycolysis by monoclonal antibody (mAb) targeting extracellular or membrane-associated ENO-1 in tumor cells of multiple myeloma was shown. In FIG. 3, human multiple myeloma cell lines RPMI-8226 and U266 were treated with 1 or 10 μg/ml of ENO-1 mAb or 10 μg/ml control IgG for 48 hours. Supernatants were collected for measurement of lactate production. Results showed targeting extracellular or cell surface ENO-1 by ENO-1 mAb could reduce lactate production (i.e. glycolysis) in multiple myeloma cells.

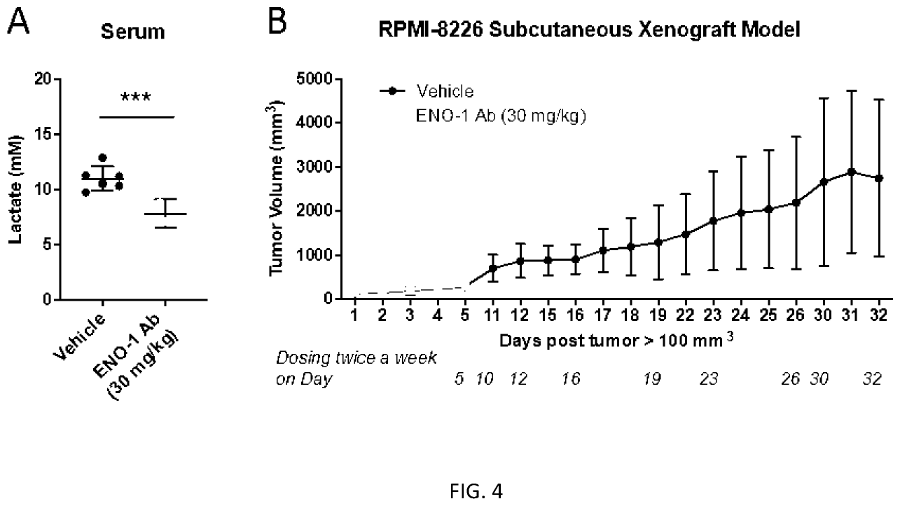

ENO-1 mAb was evaluated in multiple myeloma subcutaneous xenograft model. The 7-week-old male Balb/c nude mice were subcutaneously inoculated with human multiple myeloma RPMI-8226 cells. Mice were randomly divided into 2 groups with 6 mice in each group. The day was set as day 0 when first tumor grew over 100 mm³. Mice of ENO-1 Ab group were injected intraperitoneally with 30 mg/kg of ENO-1 mAb on day 5, 10, 12, 16, 23, 26, 30, and 32. The mice were sacrificed on day 33. Results demonstrates that treating with ENO-1 mAb was able to suppress serum levels of lactate (i.e. glycolysis) as well as tumor growth (FIG. 4).

### Example 3. In Vitro Effects of Targeting Extracellular ENO-1 by Monoclonal Antibody to Regulate Glycolysis on Prostate Cancer Cell Line PC-3

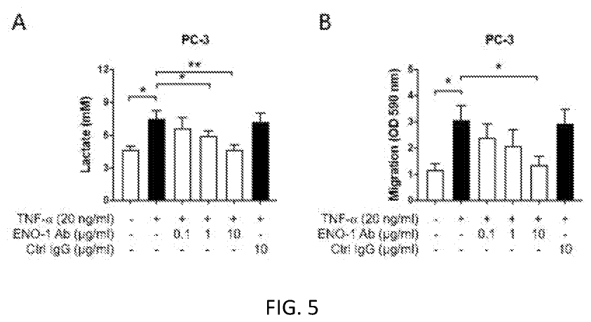

In this example, one method of regulating glycolysis by monoclonal antibody (mAb) targeting extracellular or membrane-associated ENO-1 was shown. Androgen-resistant prostate cancer cell line PC-3 was treated with 20 ng/ml TNF-α in vitro to mimic the inflammatory tumor microenvironment of prostate cancer. PC-3 cells were treated with 0.1, 1 or 10 μg/ml of ENO-1 mAb or 10 μg/ml control IgG for 48 hours. Supernatants were collected for measurement of lactate production. FIG. 5 demonstrated ENO-1 mAb dose-dependently reduced both lactate production (i.e. glycolysis) and cell migration of PC-3 cells after TNF-α induction.

### Example 4. In Vitro and In Vivo Effects of Targeting Extracellular ENO-1 by Monoclonal Antibody to Regulate Glycolysis in Pulmonary Fibrosis Model

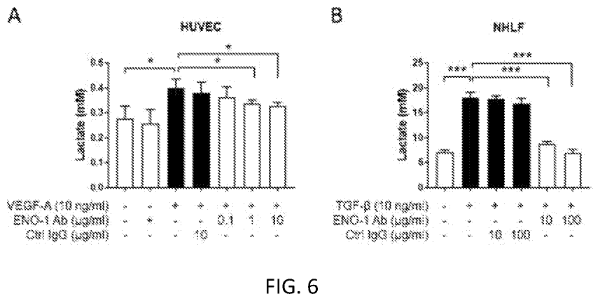

In this example, one method of regulating glycolysis by monoclonal antibody targeting extracellular or membrane-associated ENO-1 in fibrosis-associated cells, vascular endothelial cells and lung fibroblasts, was shown. Deregulated angiogenesis occurs simultaneously in pulmonary fibrosis. Migration and proliferation of endothelial cells rely on glycolysis as a fuel source during angiogenesis. In FIG. 6A, primary human umbilical vascular endothelial cells (HUVEC) were treated with 10 ng/ml VEGF-A in the absence or presence of indicated concentrations of ENO-1 mAb or control IgG for 18 hours. Supernatants were collected for measurement of lactate production. TGF-β was thought as a main driver of tissue fibrosis by inducing aberrant fibroblast activation. Metabolic shift towards glycolysis in lung fibroblasts is closely related to pulmonary fibrosis. In FIG. 6B, primary human lung fibroblasts (NHLF) were treated with 10 ng/ml TGF-β in the absence or presence of indicated concentrations of ENO-1 mAb or control IgG for 24 hours. Results illustrate targeting extracellular or cell surface ENO-1 by ENO-1 mAb could reduce lactate production (i.e. glycolysis) in both cells.

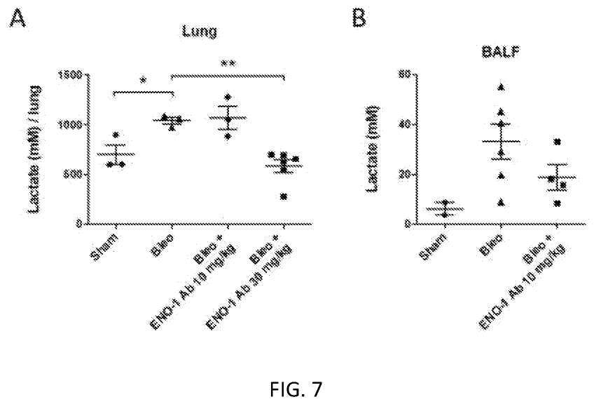

ENO-1 mAb was evaluated in a murine model of bleomycin (Bleo)-induced pulmonary fibrosis (FIG. 7). The 7-week-old male C57BL/6 mice were intratracheally given with single dosing of bleomycin (3 mg/kg). The day of bleomycin challenge was set as day 0. Mice of ENO-1 mAb treatment group were injected intravenously on day 1, 7, 13, and 19. Results illustrate treating with ENO-1 mAb was able to reduce the levels of lactate in the lungs as well as in the bronchial alveolar lavage fluids (BALF).

With above mentioned examples, it is obvious that ENO-1 mAb can reduce the levels of lactate in different situations, thereby can be used to treat different human diseases arising from aberrant activation or expression of ENO-1.

### Administering of ENO-1 Antagonist or Agonist

In some instances, ENO-1 antagonist or agonist is administered orally, subcutaneous injection, intramuscular injection, intravenous injection, intraperitoneal injection or orthotopic injection, preferably, by intravenous bolus injection, intravenous infusion or subcutaneous bolus injection.

In some instances, the effective amount of ENO-1 antagonist or agonist administered as part of the methods described herein is from 1-5000 mg/kg. However, an effective amount may vary from mammal to mammal and can easily be adjusted by one of ordinary skill by varying the amount of the active ingredient and frequency of administrations. The amount of ENO-1 antagonist or agonist administered will depend upon a variety of factors, including, e.g., the particular indication being treated, the mode of administration, the severity of the indication being treated and the age and weight of the patient, the bioavailability of the particular modality, and the like.

In some embodiments, the ENO-1 antagonist or agonist is administered to the subject intravenously (e.g., over a 30-, 60- or 120-minutes infusion, preferably 120 minutes).

### Dosing of ENO-1 Antagonist or Agonist

In some instances, the effective amount of the ENO-1 antagonist or agonist (e.g., an ENO-1 antagonist antibody as disclosed herein) is a fixed dose of between about 10 mg to about 3000 mg every 2 to 4 weeks.

With the above-mentioned disclosure, the ENO-1 antagonist or agonist can affect the progress of glycolysis, and thereby can control the growth of cancer. The present disclosure has provided a new method for treating cancer by regulating glycolysis by affecting the extracellular or membrane-associated ENO-1 of a cell.

The description is presented to enable one of ordinary skill in the art to make and use the invention. Various modifications to the described embodiments are readily apparent to those persons skilled in the art and the generic principles herein can be applied to other embodiments. Thus, the present invention is not intended to be limited to the embodiments shown but is to be accorded the widest scope consistent with the principles and features described herein. It is readily apparent to one skilled in the art that other modifications can be made to the embodiments without departing from the spirit and scope of the invention as defined by the appended claims.

What is claimed is:

1. A method for regulating glycolysis in a cell, comprising:
contacting the cell with an alpha-enolase (ENO-1) antagonist or agonist which specifically binds to extracellular or membrane-associated ENO-1 of the cell.

2. The method of claim 1, wherein the ENO-1 antagonist or agonist is an ENO-1 antibody, peptide, aptamer, small molecule compound or the binding fragment thereof.

3. Use of an alpha-enolase (ENO-1) antagonist or agonist in manufacturing a medicament for regulating glycolysis.

4. An alpha-enolase (ENO-1) antagonist or agonist for use in regulating glycolysis in a cell.

5. A method of treating human disease arising from aberrant activation or expression of an alpha-enolase (ENO-1), comprising:
administering to a subject in need a therapeutically effective amount of an ENO-1 antagonist or agonist targeting extracellular or membrane-associated ENO-1.

6. The method of claim 5, wherein the administering step is by oral, parenteral, buccal, vaginal, rectal, inhalation, insufflation, sublingual, intramuscular, subcutaneous, topical, intranasal, intraperitoneal, intrathoracic, intravenous, epidural, intrathecal, or intracerebroventricular route, or by injection into joint.

7. The method of claim 5, wherein the administering step is by intravenous bolus injection or intravenous infusion over 30, 60 or 120 minutes.

8. The method of claim 5, wherein the administering step is by subcutaneous bolus injection.

9. The method of claim 5, wherein the administering step is provided with a dosing regimen comprising one or more dosing cycles of the ENO-1 antibody at a fixed dose of about 10-3000 mg every 2 to 4 weeks.

10. The method of claim 5, wherein the human diseases comprise cancers, immune diseases, or fibrotic disease.

11. The method of claim 10, wherein the cancers comprise gastrointestinal cancer, including colon cancer, colorectal cancer, esophagus cancer, gastric cancer, hepatocellular cancer, liver cancer and pancreatic cancer, lymphoproliferative disorders, including lymphoma, lung cancer, including non-small cell lung cancer, including adenocarcinoma of the lung, squamous carcinoma of the lung, and small-cell lung cancer, blood cancer, including leukemia, bladder cancer, blastoma, brain cancer, breast cancer, cancer of the peritoneum, cervical cancer, endometrial or uterine carcinoma, glioblastoma, glioma, head and neck cancer and kidney cancer.

12. The method of claim 10, wherein the immune diseases comprise multiple sclerosis, systemic sclerosis, systemic lupus erythematosus, rheumatoid arthritis, type 1 diabetes, atherosclerosis, macrophage activation syndrome, psoriasis, atopic dermatitis, or inflammatory bowel diseases.

13. The method of claim 10, wherein the fibrotic diseases comprises idiopathic pulmonary fibrosis, pulmonary hypertension, emphysema, nonalcoholic steatohepatitis, pancreatic fibrosis, renal fibrosis, intestinal fibrosis, cardiac fibrosis, myelofibrosis, arthrofibrosis, systemic sclerosis, interstitial lung diseases, non-specific interstitial pneumonia (NSIP), usual interstitial pneumonia (UIP), endomyocardial fibrosis, mediastinal fibrosis, retroperitoneal fibrosis, progressive massive fibrosis (a complication of coal workers' pneumoconiosis), nephrogenic systemic fibrosis, Crohn's disease, old myocardial infarction, scleroderma/systemic sclerosis, neurofibromatosis, Hermansky-Pudlak syndrome, diabetic nephropathy, hypertrophic cardiomyopathy (HCM), hypertension-related nephropathy, focal segmental glomerulosclerosis (FSGS), radiation-induced fibrosis, uterine leiomyomas (fibroids), alcoholic liver disease, hepatic steatosis, hepatic fibrosis, hepatic cirrhosis, hepatitis C virus (HCV) infection, chronic organ transplant rejection, fibrotic conditions of the skin, keloid scarring, Dupuytren contracture, Ehlers-Danlos syndrome, epidermolysis bullosa dystrophica, oral submucous fibrosis, and fibro-proliferative disorders.
